(19) United States
(12) Patent Application Publication
(10) Pub. No.: US 2016/0213802 A1
(43) Pub. Date: Jul. 28, 2016

(54) DEVICE AND METHOD FOR FRAGRANCING AN INNER SPACE OF A MOTOR VEHICLE

(71) Applicant: FORD GLOBAL TECHNOLOGIES, LLC, Dearborn, MI (US)

(72) Inventors: Volker SCHEER, Roetgen (DE); Jan FASSBENDER, Koenigswinter (DE)

(21) Appl. No.: 14/997,152

(22) Filed: Jan. 15, 2016

(30) Foreign Application Priority Data
Jan. 27, 2015 (DE) 10 2015 201 364.2

(51) Int. Cl. A61L 9/03; A61L 9/02

(52) U.S. Cl. CPC A61L 9/03; A61L 9/02

(57) ABSTRACT

A vehicle system includes a container adapted to hold a fluid fragrance substance, a pump connected to the container, a capillary pipe connected to the pump and sized to retain the fluid fragrance substance within the capillary pipe when the pump is off, and an evaporation grid connected to the capillary pipe. The evaporation grid may have open porosity and be at least partially positioned within a vehicle ventilation channel to selectively provide fragrance to the vehicle.

### CROSS-REFERENCE TO RELATED APPLICATIONS

This application claims foreign priority benefits under 35 U.S.C. §119(a)-(d) to DE 10 2015 201 364.2 filed Jan. 27, 2015, which is hereby incorporated by reference in its entirety.

### TECHNICAL FIELD

The disclosure relates to a device and a method for fragrancing an inner space of a motor vehicle with at least one fragrance substance.

### BACKGROUND

Fragrance devices are known and are used to convey a pleasant sensation to vehicle occupants and thus to improve the travel comfort and well-being or also to mask bad odors or odors which are perceived to be unpleasant in the inner space of a vehicle

FR2821797A1 discloses a device for introducing at least one substance, in particular a perfume, into an inner space of a motor vehicle. The device contains a perfume tank and a pump means which is connected to the tank in order to introduce perfume drop by drop into a ventilation or heating channel of the inner space. It further contains a heating element, on which drops of perfume are evaporated, wherein the heating element is an electronic component or a cooling member which belongs to such a component. The device has the disadvantage that the metering and evaporation of the perfume, and in particular the fragrance effect produced, cannot be controlled in a precise manner. As a result of the drop-by-drop provision of the fragrance substance, a residue which is then slowly evaporated and continues to produce a fragrancing effect may remain between the pump means and the heating element, particularly after the pump means has been switched off. A change of the impression of the odor can also be brought about by selective evaporation of highly volatile components of the fragrance substance in such a residual quantity between the pump means and the heating element.

KR20060073129A describes a device which is installed in the air conditioning system of a vehicle to add an aroma substance to the incoming air for air-conditioning the vehicle. A wick conveys the aroma substance as a result of the capillary action thereof out of a container into an air line which is connected in parallel with the fan of the air conditioning system and therefore the main air guiding system, wherein the inlet opening for the wick in the air line is provided with a heating element. By the heating action of the heating element being adapted, the evaporation and therefore also the subsequent conveying of the aroma substance by the wick can be controlled. At both ends of the air line, the air flow through the air line can be adjusted. In this device, there is also produced the disadvantage that a residual quantity of aroma substance remains in the wick in the event of switching off. By means of the relatively large surface of the wick, aroma substance then also continues to be evaporated when the heating is switched off and is even subsequently conveyed via the wick. Even if an air flow of the air line which is provided separately is prevented and therefore the evaporated aroma substance is no longer introduced into the incoming air for air conditioning the vehicle, a change and a non-uniform occurrence of the impression of the odor can be produced during the action of switching on again as a result of selective evaporation and ageing of the aroma substance in the air space which is then closed.

### SUMMARY

In one embodiment a method or device for providing fragrance to a vehicle interior includes a tank for the fragrance substance and a pump connected to the tank to introduce the fragrance substance into a ventilation or heating channel of the vehicle interior.

In various embodiments a fragrance device or method for an inner space of a motor vehicle include a fragrance function that can be switched on and off in a precise manner. After switching off, in particular after a pump has been switched off, no subsequent evaporation of a residual quantity of fragrance substance is intended to occur. Furthermore, the fragrance substance is intended to be provided in a ventilation or heating channel of the inner space immediately after switching on and the evaporation is intended to be able to begin. In one or more embodiments, this is achieved with a capillary body that acts as an odor trap fitted between the pump and the interior of the ventilation or heating channel.

The term “capillary body” is intended to be understood to be a device in which, in the event of contact with a fluid, capillary forces act on this fluid so that a capillary pressure is produced. The capillary forces can be produced, for example, by a structural porosity and/or small inner cross-section and the associated capillary effect in the event of wetting with a fluid. The term “porosity in the structure of the capillary body” is intended to be understood in this instance to mean an open porosity in which small cavities are connected to each other and to the environment. The capillary pressure produced can be used to transport the fluid, wherein the transport may be simple onward transport or also a distribution into different regions.

Fragrance devices often use a fluid fragrance substance with the actual aroma substances dissolved in a carrier fluid. Fragrant oils may be mentioned by way of example. As a result of the evaporation of the carrier fluid, the aroma substances are released and distributed in the air and thus achieve the desired fragrance effect. The fluid transport from a tank to the location at which the fragrance substance is evaporated can be brought about completely or partially by a capillary effect. When a capillary is configured, the achievable capillary pressure and therefore also the ascent height which is able to be achieved counter to gravitational force in respect of the fluid is fixed by adapting the inner diameter of at least one capillary channel to the fluid to be transported, in particular to the surface tension thereof and the wetting angle.

According to various embodiments, the capillary body is configured in such a manner that the capillary action thereof is not sufficient, when the pump is switched off, to transport the fragrance substance over the entire path as far as the ventilation or heating channel of the inner space, but it nevertheless prevents the fragrance substance from flowing back into the pump or the tank. It can thereby act as an odor trap of the fragrance device. The transport of the fragrance substance from the tank to the capillary body and as far as the location from which the capillary effect can be used for onward transport is carried out by a pump. If the pump is not operational, the capillary body produces a fluid level which levels off at a location where no significant subsequent evaporation of the fragrance substance or components of the fragrance substance can occur as a result of a very small free surface which is in contact with air. The function as an odor trap is thereby possible, that is to say, no fragrance or odor is produced when the pump is not operational. The capillary effect further prevents the fragrance substance from flowing back into the pump or the tank, which is advantageous in the event of switching back on because the fragrance substance can thus be provided in a ventilation or heating channel of the inner space directly after the switching-on action.

To achieve more effective evaporation of the fragrance substance in the ventilation or heating channel of the inner space, it is advantageous to at least partially heat the capillary body so that the fragrance substance evaporates at a higher partial saturation pressure, where it can better be taken up by the air.

It is also advantageous for the capillary body to be a capillary compound, that is to say, an assembly comprising a capillary pipe and a porous evaporation element which is formed in a planar manner and which is acted on by an air flow within the ventilation or heating channel. The term “planar” is intended to mean in this instance that the evaporation element has, in relation to its volume, a very large outer surface which can be used for the effective evaporation of the fragrance substance. The capillary pipe can then act as an odor trap when the pump means is switched off because the very small inner cross-sectional surface area of the capillary pipe allows only a very small free surface of the fluid fragrance substance, via which surface no relevant quantity of fragrant substances can be released. The evaporation element having a large surface ensures the efficient distribution and evaporation of the fragrance substance in the evaporation or heating channel when the pump is operational.

If the evaporation element is constructed in this case in such a manner that it can be completely passed through by the air flow, a residue-free evaporation of a quantity of the fragrance substance located in the evaporation element into the air flow can then be brought about. This is advantageous to prevent additional production of odors from a residual quantity of the fragrant substance still remaining immediately after the fragrance function is switched off.

It is further advantageous for the evaporation element to be an electrical heating element. This allows direct heating of the evaporation element by application of an electrical voltage. An electrical current through the evaporation element is produced, wherein the electrical resistance of the evaporation element produces a power loss which occurs in the form of heat. The heating power can then be adjusted with the voltage. The heating power produced at a specific voltage can be adapted, for example, by variation of material selection or material thickness or, for example, a wire cross-section. The self-heating of the evaporation element has the particular advantage that the heating action can be switched on and off very quickly and no additional thermal capacities have to be heated.

It is also advantageous for the evaporation element to contain a grid which constitutes a small resistance for the air flow in the ventilation or heating channel, particularly if the fragrance function is switched off and the evaporation element is not charged with the fragrance substance. The air throughput in the ventilation or heating channel is influenced as little as possible. The production of air flow noise can also be reduced or prevented by the evaporation element being constructed as a grid. Furthermore, the capillary effect in a tightly meshed grid allows efficient distribution of the fragrance substance on the grid surface.

In a similar manner, the evaporation element can also be constructed as a honeycomb structure, where rectification of the air flow and/or reduction of flow noise can simultaneously be achieved in the ventilation or heating channel.

A fragrance device according to embodiments of the present disclosure may be advantageously operation using a method in which the pump for conveying the fragrance substance is switched on when fragrance for the inner space of the motor vehicle is intended to be carried out so that the fragrance substance is introduced by the capillary body into the ventilation or heating channel. If fragrance for the inner space of the motor vehicle is not intended to be carried out, however, the pump conveying the fragrance substance is switched off so that the flow of the fragrance substance is stopped and the fragrance substance inside the capillary body remains at a location where, as a result of a very small, free surface which is in contact with air, no significant subsequent evaporation of the fragrance substance or components of the fragrance substance occurs and the capillary body consequently acts as an odor trap.

It is also advantageous for the capillary body to be heated or partially heated in the method according to various embodiments when fragrance for the inner space of the motor vehicle is intended to be carried out so that the fragrance substance can be evaporated more effectively. When fragrance for the inner space of the motor vehicle is not intended to be carried out, the heating or partial heating of the capillary body can be switched off as soon as the fragrance substance in the heated region of the capillary body is evaporated so that no significant subsequent evaporation of a residual quantity of the fragrance substance or components of the fragrance substance occurs.

### BRIEF DESCRIPTION OF THE DRAWINGS

FIG. 1 illustrates operation of a method or device for providing fragrance to a vehicle interior according to various embodiments.

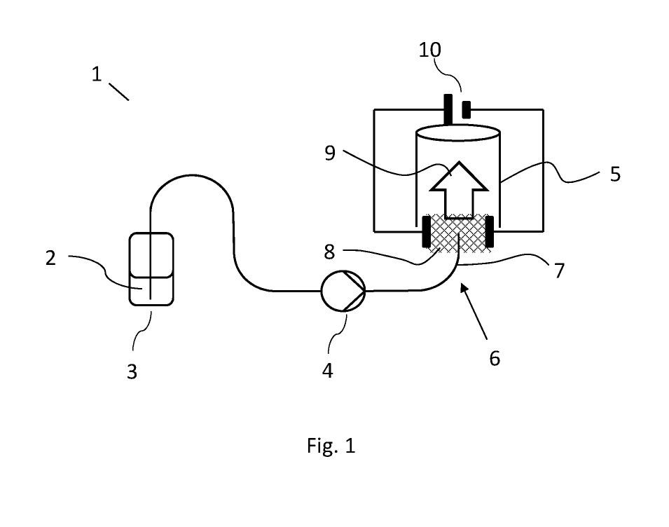

### DETAILED DESCRIPTION

As required, detailed embodiments are disclosed herein; however, it is to be understood that the disclosed embodiments are merely illustrative and may be embodied in various and alternative forms. The figure is not necessarily to scale; some features may be exaggerated or minimized to show details of particular components. Therefore, specific structural and functional details disclosed herein are not to be interpreted as limiting, but merely as a representative basis for teaching one skilled in the art to variously employ the claimed subject matter.

The device 1 for fragrancing the inner space of a motor vehicle contains a fragrance substance 2 which is stored in fluid form in a tank 3. A pump 4, which may be a pump driven by an electric motor, provides the fragrance substance 2 under pressure in a capillary body 6 which projects into a ventilation or heating channel of the inner space 5 of the motor vehicle. The capillary body 6 comprises a capillary pipe 7 and an evaporation element 8. The pressure produced by the pump 4 allows it, together with the capillary action of the capillary pipe 7, to transport the fragrance substance 2 into the evaporation element 8. In this instance, the capillary pipe 7 is configured in such a manner that the ascent height h which can be achieved by the capillary action above the filling level of the fragrance substance 2 in the tank 3 is not by itself sufficient to convey the fragrance substance 2 without operation of the pump 4. The ascent height h results from:

$$h = \frac{2 σ cos θ}{ρ g r}$$

where:
h represents ascent height,
σ represents surface tension,
θ represents contact angle,
ρ represents fluid density,
g represents gravitational acceleration, and
r represents the radius of the capillary pipe.

The fragrance substance 2 is extensively distributed by capillary action and evaporated into an air flow 9 on the evaporation element 8 which is constructed according to one embodiment as an electrical heating element in the form of a grid. To that end, the evaporation element is flowed through by a current which is subjected to resistance by an electrical voltage 10 being applied so that the element becomes heated by the electrical power loss and promotes the evaporation of the fragrance substance 2.

While representative embodiments are described above, it is not intended that these embodiments describe all possible forms of the claimed subject matter. Rather, the words used in the specification are words of description rather than limitation, and it is understood that various changes may be made without departing from the spirit and scope of the disclosure. Additionally, the features of various implementing embodiments may be combined to form further embodiments that may not be explicitly described or illustrated.

What is claimed is:

1. A device providing fragrance to an inner space of a motor vehicle, comprising:
a tank configured to hold a fragrance substance;
a pump connected to the tank and configured to introduce the fragrance substance into a ventilation or heating channel of the inner space; and
a capillary body disposed between the pump and the interior of the ventilation or heating channel, the capillary body acting as an odor trap.

2. The device as claimed in claim 1, wherein the capillary body is configured to be at least partially heated by an electric current flowing through at least a portion of the capillary body.

3. The device as claimed in claim 1, the capillary body including a capillary compound comprising:
a capillary pipe; and
a planar evaporation element having open porosity and positioned to be acted on by air flow within the ventilation or heating channel of the inner space.

4. The device as claimed in claim 3, the evaporation element comprising an electrical heating element.

5. The device as claimed in claim 4, the evaporation element comprising a grid.

6. The device as claimed in claim 4, the evaporation element comprising a honeycomb structure.

7. The device as claimed in claim 1, the capillary body being positioned higher than the tank and having an opening sized to retain the fragrance substance within the capillary body against gravitational flow to the tank when the pump is switched off.

8. A method for providing fragrance to an inner space of a motor vehicle using a fragrance substance, comprising:
pumping, in response to a request for providing fragrance to the inner space of the motor vehicle, the fragrance substance from a tank through a capillary body positioned at least partially within a ventilation channel of the motor vehicle.

9. The method of claim 8, further comprising heating at least a portion of the capillary body by applying a voltage or current to the capillary body in response to the request.

10. The method of claim 8, further comprising heating the capillary body in response to the request.

11. The method of claim 8, further comprising positioning the capillary body higher than the tank.

12. The method of claim 8, the capillary body positioned higher than the tank and including an opening configured to retain the fragrance substance within the capillary body against gravity when the pumping stops.

13. The method of claim 8, the capillary body comprising:
a capillary pipe connected to the pump and having an opening configured to retain the fragrance substance within the capillary pipe against gravity when the pumping stops; and
an evaporation element connected to the capillary pipe and positioned at least partially within the ventilation channel.

14. The method of claim 8, wherein the pumping comprises pumping the fragrance substance through a capillary pipe of the capillary body and into a planar evaporation element of the capillary body.

15. A vehicle system comprising:
a container adapted to hold a fluid fragrance substance;
a pump connected to the container;
a capillary pipe connected to the pump and sized to retain the fluid fragrance substance within the capillary pipe when the pump is off; and
an evaporation grid connected to the capillary pipe, having open porosity, and at least partially positioned within a vehicle ventilation channel to selectively provide fragrance to the vehicle.

16. The vehicle system of claim 15, the evaporation grid comprising a honeycomb structure.

17. The vehicle system of claim 15, the evaporation grid configured to heat the fluid fragrance substance in response to an applied voltage.

18. The vehicle system of claim 15, the capillary pipe having an opening connecting to the evaporation grid and positioned higher than the container.

19. The vehicle system of claim 15, the evaporation grid comprising a planar grid.

20. The vehicle system of claim 19, the planar grid comprising a honeycomb structure.
